(19) United States
(12) Patent Application Publication
(10) Pub. No.: US 2011/0280803 A1
(43) Pub. Date: Nov. 17, 2011

(54) RADIOIODINATION METHOD

(75) Inventors: Michelle Avory, Amersham (GB); Jane Brown, Amersham (GB); Peter Brian Iveson, Amersham (GB)

(73) Assignee: GE HEALTHCARE UK LIMITED, LITTLE CHALFONT (GB)

(21) Appl. No.: 13/145,136

(22) PCT Filed: Jan. 29, 2010
(86) PCT No.: PCT/EP2010/051053

§ 371 Date: Jul. 19, 2011

Related U.S. Application Data
(60) Provisional application No. 61/148,410, filed on Jan. 30, 2009.

(30) Foreign Application Priority Data
Jan. 29, 2009 (GB) 0901483.8

(51) Int. Cl. A61K 51/04; C07C 231/02; B65D 85/00; C07C 259/06; A61P 43/00; C07D 213/77; C07D 213/64

(52) U.S. Cl. 424/1.85; 546/306; 564/134; 546/291; 562/621; 206/216

(57) ABSTRACT

The present invention provides a method for the synthesis of radioiodinated compounds which is advantageous over prior art methods. Using a hydrazine or an aminoxy in place of a primary amine for indirect radioiodination facilitates a much quicker reaction thus reducing reaction time and increasing the yield. In addition, where there are primary amines in the molecule to be radioiodinated, such as the N-terminus of a peptide or lysine residues, reaction at the hydrazine or aminoxy is greatly favoured.

### TECHNICAL FIELD OF THE INVENTION

The present invention relates to the synthesis of radioiodinated compounds, and in particular to indirect radioiodination methods. The method of the present invention provides advantages over presently-known indirect radioiodination methods.

### DESCRIPTION OF RELATED ART

Radioiodination is most easily carried out via direct radioiodination by reacting radioiodine with a suitable precursor compound. For example, the precursor compound may comprise: an aryl iodide or bromide (to permit radioiodine exchange); an activated precursor compound aryl ring (e.g. a phenol group); an organometallic precursor compound (e.g. trialkyltin, trialkylsilyl or organoboron compound); or an organic precursor compound such as triazenes or a good leaving group for nucleophilic substitution such as an iodonium salt.

Although simple to perform, direct radioiodination has disadvantages, especially when applied to the radioiodination of biomolecules such as proteins. For example, radioiodination of proteins by direct electrophilic substitution of hydroxyl groups on tyrosine residues cannot be applied to proteins lacking tyrosine residues. Furthermore, proteins radioiodinated by electrophilic methods often exhibit reduced biological function resulting from direct exposure to oxidising conditions. Another problem with direct radioiodination is that is that proteins labelled in this way often undergo in vivo deiodination due to the structural similarity of iodotyrosine residues with thyroid hormones. To address these problems, indirect radioiodination methods have been developed. The known indirect radioiodination methods comprise formation of a radioiodinated synthon, which is then conjugated to a protein under mild conditions through modification of lysine ε-amino groups (for a review see Wilbur 1992 Bioconj Chem; 3: 433-70). Currently the most commonly used indirect radioiodination method involves conjugation of an iodo Bolton-Hunter reagent, or more typically N-succinimidyl-3-iodo-benzoate (SIB), to a primary amine present in the vector, as illustrated in scheme 1 below:

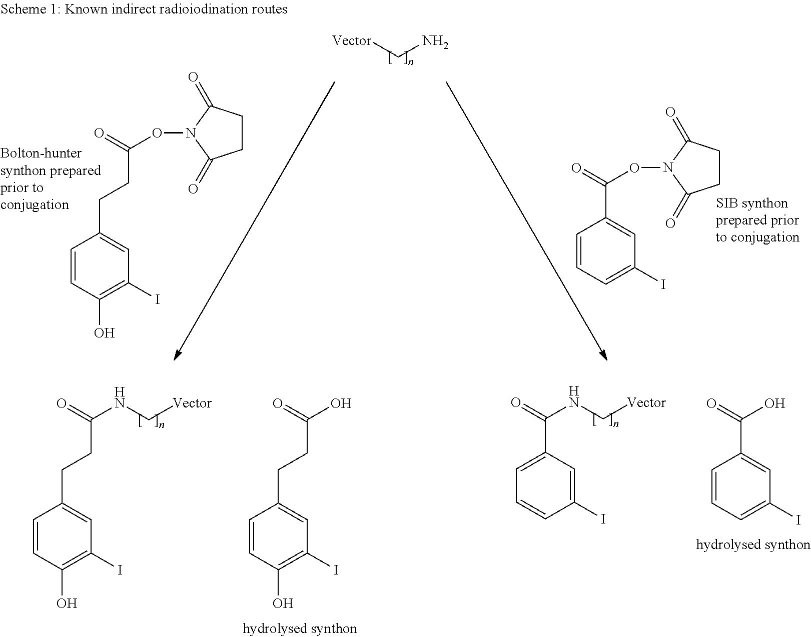

A typical such reactions takes 30 minutes at 37° C., pH 7.5-8, 50 mM, in sodium phosphate buffer. The reaction generally proceeds more quickly at higher pH. However, as the pH is increased so the hydrolysis of the NHS ester increases and the subsequent yields can be compromised for that step. The yields also vary depending on the vector, its solubility, position of the amine functionality and the system chosen for the conjugation.

The formation of hydrazones by the action of hydrazine on ketones or aldehydes is known (March's Advanced Organic Chemistry 5^{th} Edition, 2001, by Smith and March; Chapter 16 pp 1192-4), and can be used for the chemoselective coupling of a carbonyl functionality to a hydrazine-functionalised compound. In the art of radiochemistry, the hydrazine-functionalised compound 6-hydrazinonicotinic acid (HYNIC) is a well-known bifunctional ligand for ^{99m}Tc labelling of biomolecules (see for example Abrams et al 1990 J Nuc Med; 31. 2022-8). HYNIC has also more recently been reported as an attractive route to label biomolecules with ¹⁸F. Chang et al (2005 Bioconj Chem; 16: 1329-33) describe the preparation of ¹⁸F labelled human serum albumin (HSA) by conjugation of [¹⁸F]fluorobenzaldehyde ([¹⁸F]FBA) with HYNIC-HSA via hydrazone formation. The same group also reported application of this method in the preparation of an ¹⁸F labelled RGD peptide (Lee et al 2006 Nuc Med Biol; 33: 677-83). Bruus-Jensen et al (2006 Nuc Med Biol; 33: 173-83) investigated the method in the formation of various other ¹⁸F labelled peptides and reported that it was a fast and straightforward radiolabeling method leading to high yields under mild acidic conditions. In these reports, it was found that the optimum temperature for hydrazone formation ranged between 50-70° C. This temperature range is not the most ideal for reactions comprising biomolecules, which are ideally carried out at temperatures closer to physiological temperature.

There is scope to provide improved methods for the radioiodination of biomolecules.

### SUMMARY OF THE INVENTION

The present invention provides a method for the synthesis of radioiodinated biomolecules which is advantageous over presently-known methods. Using reaction between a hydrazine or an aminoxy derivative and an active ester facilitates a much quicker reaction thus reducing reaction time and increasing the yield as compared with the known reaction between an amine and an active ester. In addition, where there are primary amines in the molecule to be radioiodinated, such as the N-terminus of a peptide or lysine residues, reaction at the hydrazine or aminoxy group is greatly favoured. Furthermore, the method of the invention provides good yields at temperatures that mimic those of the native state of the biomolecule. This provides the advantage of optimal preservation of the biomolecule's structure and function.

### DETAILED DESCRIPTION OF THE INVENTION

### Method for Synthesis of a Radioiodinated Compound

In one aspect, the present invention relates to a method for the synthesis of a radioiodinated compound of Formula I:

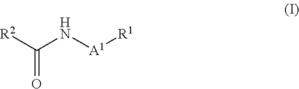

or a salt or solvate thereof, said method comprising reaction of a compound of Formula II:

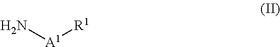

with a compound of Formula III:

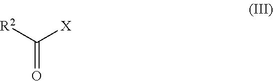

wherein:
A¹ is either NH or O;
one of R¹ and R² is the group -L¹-Ar¹ wherein:
L¹ is a bond or is a bivalent linker comprising 1-3 L* linker units wherein L^{*} is selected from —CO—, —CR′₂—, —CR′═CR′—, —C≡C—, —CR′₂CO₂—, —CO₂CR′₂—, —NR′—, —NR′CO—, —CONR′—, —NR′—, —(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₅₋₁₂ arylene group, and a C₃₋₁₂ heteroarylene group, wherein R′ is hydrogen or C₁₋₃ alkyl; and,
Ar¹ is a 6-membered C₃₋₆ aryl group, substituted with radioiodine, and with 0-3 other substituents selected from C₁₋₃ alkyl, halo, amino, carboxyl, hydroxyl, or protected versions thereof, and wherein said aryl group has 0-3 heteroatoms selected from N, S and O;
   and the other of R¹ and R² is the group -L²-R*, wherein:
L² is a bond or is a bivalent linker comprising 1-6 L* linker units wherein L* is as defined for L¹; and,
R* is a biomolecule;
   wherein R¹ and R² optionally comprise suitable protecting groups;
   and wherein X represents an active ester group.

### Definitions for Method of the Invention

The term “radioiodinated compound” means a compound comprising radioiodine, i.e. at least one radioiodine atom, and specifically in the context of the present invention, a compound comprising a radioiodoaryl group. A “radioiodoaryl group” is an aryl group, as defined herein, comprising a radioiodine atom. A “radioiodine atom” may be any radioactive isotope of iodine, preferably ¹²³I, ¹²⁴I, ¹²⁵I, or ¹³¹I, and most preferably ¹²³I, ¹²⁴I, or ¹³¹I.

Suitable salts according to the phrase “salt or solvate thereof” used in the present invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

In its broadest sense, the term “reaction of” refers to mixing a solution of the compound of Formula II with a solution of the compound of Formula III. The solutions may be aqueous or organic. An “aqueous” solution is a solution wherein the solvent comprises water. The term “organic” when referring to a solution refers to a solution wherein the solvent is a carbon-containing solvent, including such solvents as tetrahydrofuran (THF), dichloromethane (DCM) and dimethylformamide (DMF). Typically, mixing a solution of the compound of Formula II with a solution of the compound of Formula III is carried out at a defined temperature over a defined period of time. It is preferred that the method of the invention is carried out at a temperature less than 50° C., but not significantly less than room temperature. A preferred temperature for carrying out the method of the invention is therefore in the range 15-45° C., most preferably in the range 20-40° C. and especially preferably 35-40° C. An advantage of the present invention over the prior art methods is that it can successfully be carried out at 37° C.

An “active ester” is an ester made with an alcohol whose structure allows it to be easily displaced by nucleophiles such as amines, forming a stable linkage. In a preferred embodiment, the active ester is selected from N-hydroxysuccinimidyl (NHS) ester, pentafluorophenyl ester, and hydroxybenzotriazole (HOBT) ester. A most preferred active ester is NHS ester.

The term “alkyl” used either alone or as part of another group is defined herein as any straight, or branched saturated or unsaturated CₙH₂ₙ₊₁ group, wherein unless otherwise specified n is an integer between 1 and 10. Alkyl groups include for example methyl, ethyl, propyl, isopropyl, butyl, isobutyl, t-butyl, 1-methylpropyl, pentyl, isopentyl, sec-pentyl, hexyl, heptyl, and octyl.

The term “cycloalkyl” refers to an alkyl group as defined herein which is cyclic.

The term “aryl” is defined herein as any mono-, bi- or tri-cyclic C₅₋₁₄ molecular fragment or group comprising at least one aromatic ring, and preferably having 5 to 6 ring members in each ring. The term aryl embraces purely aromatic radicals such as phenyl, naphthyl, tetrahydronaphthyl, indane, and biphenyl, as well as radicals comprising at least one aromatic ring fused with one or more cycloalkyl or heterocycloalkyl rings.

The term “amino” means an —NH₂ group.

The term “hydroxyl” means an —OH group.

The term “nitro” means an —NO₂ group.

The term “halo” relates to a halogen atom selected from iodine, fluorine, bromine and chlorine.

The term “carboxyl” means a —COOH group.

The term “heteroatom” refers to any atom in a hydrocarbon moiety that is not carbon or hydrogen. In the context of the present invention, heteroatoms are selected from nitrogen, oxygen and sulfur.

The term “biomolecule” as used herein means a component or product of a cell such as a peptide, protein, antibody, carbohydrate, lipid, or nucleic acid, or synthetic versions thereof. Because they are subject to denaturation, e.g. at high temperatures or due to strongly acidic or basic conditions, biomolecules are advantageously radioiodinated indirectly and at temperatures as close to physiological temperature as possible.

The term “physiological temperature” is taken herein to refer most specifically to temperatures in the range 20-40° C. and preferably 35-40° C.

The term “alkylene”, used either alone or in combination, refers to a straight or branched chain or cyclic bivalent aliphatic radical having a specified number of carbon atoms. Examples of alkylenes as used herein include, but are not limited to, methylene, ethylene, propylene, butylene and the like.

The term “cycloalkylene” refers to a cyclic bivalent aliphatic radical.

The term “arylene”, used either alone or in combination, refers to bivalent unsaturated aromatic carboxylic radicals having a single ring, such as phenylene, or multiple condensed rings, such as naphthylene or anthrylene. Examples of arylenes as used herein include, but are not limited to, benzene-1,2-diyl, benzene-1,3-diyl, benzene-1,4-diyl, naphthalene-1,8-diyl, and the like.

The term “heteroarylene” refers to an arylene group comprising one or more heteroatoms selected from N, S or O.

A “suitable protecting group” is a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. The use of further protecting groups are described in ‘Protective Groups in Organic Synthesis’, Theorodora W. Greene and Peter G. M. Wuts, (Fourth Edition, John Wiley & Sons Inc., 2007).

### How to Obtain Compounds of Formulas II and III

The compounds of Formulas II and III may be obtained by methods known to the person skilled in the art.

To obtain a compound of either Formula II or Formula III comprising the group -L¹-Ar¹, i.e. comprising a radioiodoaryl group, a starting compound can be used that comprises a derivative which either undergoes electrophilic or nucleophilic radioiodination or undergoes condensation with a labelled aldehyde or ketone. Examples of the first category are:
(a) organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates);
(b) aromatic rings activated towards electrophilic iodination (e.g. phenols) and aromatic rings activated towards nucleophilic iodination (e.g. aryl iodonium salt aryl diazonium, aryl trialkylammonium salts or nitroaryl derivatives).

The starting compound preferably comprises: an aryl iodide or bromide (to permit radioiodine exchange); an activated precursor compound aryl ring (e.g. a phenol group); an organometallic precursor compound (e.g. trialkyltin, trialkylsilyl or organoboron compound); or an organic precursor compound such as triazenes or a good leaving group for nucleophilic substitution such as an iodonium salt. Precursor compounds and methods of introducing radioiodine into organic molecules are described by Bolton (J. Lab Comp Radiopharm 2002; 45: 485-528). Precursor compounds and methods of introducing radioiodine into proteins are described by Wilbur (Bioconj Chem 1992; 3(6): 433-470). Suitable boronate ester organoboron compounds and their preparation are described by Kabalaka et al (Nucl Med Biol, 2002; 29: 841-843 and 2003; 30: 369-373). Suitable organotrifluoroborates and their preparation are described by Kabalaka et al (Nucl Med Biol, 2004; 31: 935-938). Preferred starting compounds for radioiodination comprise an organometallic precursor compound, most preferably a trialkyltin.

Examples of aryl groups to which radioiodine can be attached are given below:

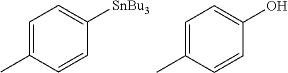

Both contain substituents which permit facile radioiodine substitution onto the aromatic ring to form the group Ar¹. Ar¹ substituents containing radioactive iodine can be synthesised by direct iodination via radiohalogen exchange, e.g.

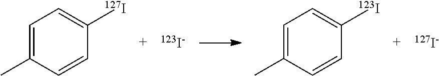

In one embodiment, A¹ of Formula II is NH, such that the compound of Formula II is a hydrazine. A wide variety of hydrazine compounds are readily available commercially that can be converted to compounds of Formula II wherein A¹ is NH using standard methods of organic chemistry. For example, iodophenylhydrazine is commercially available, which can be converted by radioiodine exchange to a compound of Formula II wherein A¹ is NH, and R¹ is radioiodophenyl. Also, commercially-available hydrazino-benzoic acid may be readily converted into hydrazine compounds of Formula II, e.g.:

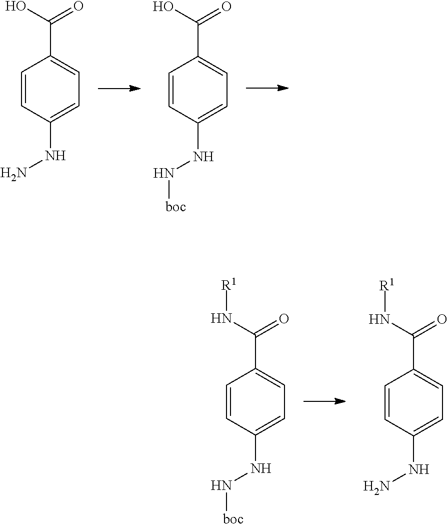

wherein boc is a tert-butoxycarbonyl protecting group, and R¹ is as defined herein.

Where R¹ is -L²-R*, the route for obtaining the compound of Formula II has to be sufficiently mild in order to be suitable for functionalisation without loss of function of the biomolecule. A route similar to that depicted above wherein the starting hydrazine-containing compound is HYNIC is well-known in the art of radiochemistry for the functionalisation of biomolecules with hydrazine. An advantage of the HYNIC moiety for introduction of a hydrazine functional group is that it can be conjugated to a biomolecule relatively easily and under mild conditions. For example, N-hydroxysuccinimidyl-HYNIC can be used to treat lysine residues in a peptide or protein (Rennen et al 2000 Nuc Med Biol; 27: 599-604), or HYNIC-malemide can be used by reacting the malemide group with cysteine residues (Banerjee et al 2005 Dalton Trans; 24: 3886-97).

An early report using a HYNIC moiety for ^{99m}Tc labelling of biomolecules (Abrams et al 1990 J Nuc Med; 31: 12) describes how to incorporate a hydrazine group into IgG by conjugating HYNIC to IgG. The resultant nicotinyl hydrazine-modified IgG can be conjugated with ^{99m}Tc-glucoheptonate, such that the end nitrogen of the hydrazine acts to complete co-ordination of ^{99m}Tc. Others have applied similar methods for ^{99m}Tc labelling of other proteins, and of peptides, demonstrating the versatility of this approach. Blankenberg et al (1998 PNAS; 95(11) 6349-54) describe ^{99m}Tc labelling of annexin V, Rennen et al (2004 Chest; 126(6): 1954-61) describe ^{99m}Tc labelling of interleukin-8, Oyen et al (2000 Eur J Nuc Med; 27: 392-9) describe ^{99m}Tc labelling of the glycoprotein IIa/IIIb receptor antagonist DMP444, and Faintuch et al (2005 Synthesis and Reactivity in Inorganic, Metal-Organic, and Nano-Metal Chemistry; 35(1): 43-51) describe ^{99m}Tc labelling of Bombesin₇₋₁₄ NH₂.

HYNIC groups can be site-specifically conjugated to peptides by immobilising the protected peptide on a solid phase, coupling HYNIC, deprotecting the peptide and then cleaving the deprotected peptide from the solid support (Surfraz et al 2007 J Med Chem; 50: 1418-22).

HYNIC has also been reported in a route for ¹⁸F labelling of biomolecules, see e.g. Lee et al (2006 Nuc Med Biol; 33: 677-83); and, Bruus-Jensen at al (2006 Nuc Med Biol; 33: 173-83). These publications disclose functionalisation of a biomolecule with HYNIC. The hydrazine group of the HYNIC-functionalised biomolecule reacts with the aldehyde of ¹⁸F-fluorobenzaldehyde to form a stable hydrazone bond, thereby providing an ¹⁸F-labelled biomolecule.

In a preferred embodiment of the method of the invention, A¹ in the compound of Formula II is NH, and R¹ is the group -L²-R*. A preferred biomolecule in the context of the present invention is a peptide, a protein, or an antibody. The terms “peptide”, “protein” and “antibody” have their common meaning in the art, as now briefly described. A peptide is a short polymer formed from the linking, in a defined order, of amino acids, wherein one amino acid residue is linked to the next by an amide bond. Conventionally, peptides are regarded as comprising up to 50 amino acid residues. Proteins are polypeptide molecules or consist of multiple polypeptide subunits. Antibodies, also known as immunoglobulins, are gamma globulin proteins that are used by the immune systems of vertebrates.

In another embodiment, A¹ of Formula II is O, such that the compound of Formula II is an aminoxy compound. Like hydrazines, aminoxy compounds are hypernucleophiles and provide the advantage over amino compounds of being more reactive. Some aminoxy compounds are available commercially that can act as starting points for obtaining a variety of compounds of Formula II wherein A¹ is O, e.g. L-alpha-Aminoxy-beta-phenylpropionic acid hydrobromide (Apollo Scientific Ltd.), aminooxyisobutyric acid hydrochloride (Fine and Performance Chemicals Ltd.).

The prior art describes how to obtain aminoxy analogues of peptide compounds by replacement of an amino group with aminoxy (see for example Briggs and Morely 1979 JCS Perkin I: 2138-43; Yang et al 2001 J Org Chem; 66(22): 7303-12; Yang et al 1999 J Am Chem Soc; 121 (3): 589-590). Aminoxy-functionalised lipids are reported by Perouzel et al (2003 Bioconj Chem; 14: 884-98).

The compound of Formula III is an active ester. Where R² is -L¹-Ar¹ the compound of Formula III is a radioiodoaryl functionalised with an active ester. Where R² is -L²-R* the compound of Formula III is a biomolecule functionalised with an active ester.

Many active esters are readily available commercially. Conversion of a carboxylic acid to an active ester may be carried out by reaction with carbodiimide/phenol or transesterification reagent, e.g. pentafluorophenyl trifluoroacetate or p-nitrophenyl trifluoroacetate. It is well-known in the art to use active esters in the synthesis of peptide compounds (see for example Grant “Synthetic Peptides” 2002; Oxford University Press: 137-139; Anderson et al 1963 J Am Chem Soc; 85: 3039). A preferred active ester of the present invention is N-Hydroxysuccinimide (NHS) ester.

In a preferred embodiment, the compound of Formula III comprises -L¹-Ar¹, and such compounds are known in the art of indirect radioiodination. Preferably, L¹ is a bond or is a C₁₋₃ alkylene linker. Ar¹ is preferably radioiodophenyl or radioiodophenol, i.e. phenyl substituted with radioiodine and optionally substituted with hydroxyl.

Indirect radioiodination methods typically involve conjugation of an iodine moiety functionalised with an NHS ester with a primary amine. For example, Bolton and Hunter (1973 Biochem J; 133: 529-39) describe labelling of proteins with ¹²⁵I by reaction of ¹²⁵I iodinated 3-(4-hydroxyphenyl)proprionic acid NHS ester (illustrated below) with free amino groups in the protein molecule.

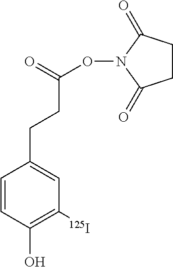

Koziorowski et al (1998 Appl Radiat Isot; 49(8): 955-9) teach how to obtain radioiodinated NHS 3- and 4-iodobenzoate (illustrated below, wherein ^{*}I is radioiodine), which are commonly-used indirect radioiodination agents.

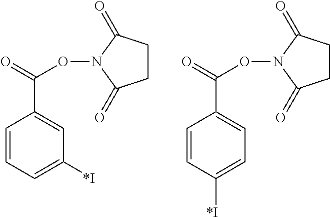

Another protocol involves reacting the free amino groups of a protein with N-succinimidyl 4-guanidinomethyl-3-[¹²⁵I]iodobenzoate (illustrated below; Vaidyanathan & Zalutsky 2007 Nature Protocols; 2: 282-6).

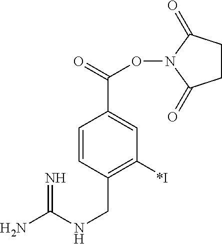

The above-described radioiodinated NHS ester compounds are preferred for use in the method of the present invention as compounds of Formula wherein R² is -L-Ar¹.

In a preferred embodiment, L¹ and L² are independently either a bond or a C₁₋₃ alkylene linker.

### Preferred Compounds

In a preferred embodiment, said compound of Formula I is a compound of Formula Ia:

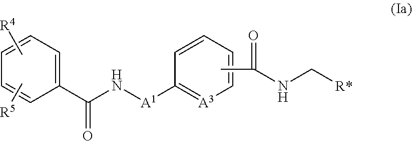

said compound of Formula II is a compound of Formula IIa:

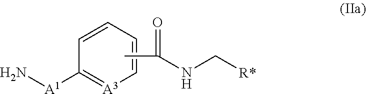

said compound of Formula III is a compound of Formula IIIa:

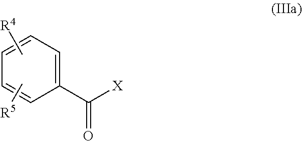

wherein A³ is N or CH, R* is as defined above, one of R⁴ and R⁵ is radioiodine and the other of R⁴ and R⁵ is hydrogen or hydroxyl, and X is an active ester as defined above.

Preferably, A³ is CH, which provides the advantage that the reaction can proceed more specifically at the A¹-NH₂ due to the absence of a heteroatom on the aryl ring. This is because the nitrogen heteroatom withdraws electrons from the hydrazine NH₂ making this less reactive towards the active ester. When A³ is CH, i.e. the ring is a phenyl ring, the electron withdrawing effect will not be exerted.

In a preferred embodiment, the method of the invention is used to obtain a radiopharmaceutical composition (described as a separate aspect of the invention below). In this case, the method of the invention further comprises one or more of the following steps:
removal of any protecting groups from the radioiodinated compound of Formula I; and/or,
(ii) admixing the radioiodinated compound of Formula I with a biocompatible carrier to form a radiopharmaceutical composition; and/or,
(iii) sterilisation of the radiopharmaceutical composition of step (ii); and/or
(iv) depyrogenation of the radiopharmaceutical composition of step (ii).

Removal of protecting groups may be carried out by methods well-known to the person skilled in the art (Greene and Wuts, supra).

Methods for sterilisation and depyrogenation are also well-known to the skilled person.

“Sterilisation” refers to any process that effectively kills or eliminates transmissible agents (such as fungi, bacteria, viruses, spore forms, etc.) from a surface, equipment, article of food or medication, or biological culture medium. Sterilisation can be achieved through application of heat, chemicals, irradiation, high pressure or filtration.

“Depyrogenation” refers to the removal of pyrogens from solution, most commonly from injectable pharmaceuticals. A “pyrogen” is defined as any substance that can cause a fever, e.g. the bacterial substance lipopolysaccharide (LPS), present in the cell wall of some bacteria, is a pyrogen. Depyrogenation may be achieved through filtration, distillation, chromatography, or inactivation.

### Automated Synthesis

In a preferred aspect, the method of the invention, as suitably and preferably defined herein, is automated. Automated processes are particularly useful in the synthesis of radioactive compounds as radiation exposure to the operator is reduced. PET radiotracers in particular are now often conveniently prepared on an automated radiosynthesis apparatus. There are several commercially-available examples of such apparatus, including TRACERlab™ and FASTlab™ (both from GE Healthcare). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps.

### Radioiodinated Compounds

The radioiodinated compound of Formula I, as suitably and prefereably defined herein for the method of the invention, is an in vivo imaging agent wherein the radioiodine substituent of Ar¹ comprises ¹²³I, ¹²⁴I or ¹³¹_{I}. By the term “in vivo imaging agent” is meant a compound designed to target a particular physiology or pathophysiology in a mammal, and which can be detected following its administration to the mammalian body in vivo. When the radioiodine-containing compound obtained by the method of the present invention is an in vivo imaging agent, the iodine atom in the radioiodoaryl moiety is selected from ¹²³I, ¹²⁴I, and ¹³¹I. The isotopes ¹²³I and ¹³¹I emit gamma rays, which can be detected using single-photon emission tomography (SPECT). The isotope ¹²⁴I emits positrons, which can be detected using positron emission tomography (PET). Preferred isotopes of iodine for the in vivo imaging agent of the invention are ¹²³I and ¹²⁴I, most preferably ¹²³I.

### Radiopharmaceutical Composition

The radioiodinated compound obtained by the method of the invention can be used for the preparation of a radiopharmaceutical composition, comprising the radioiodinated compound of Formula I as defined herein together with a biocompatible carrier suitable for mammalian administration.

A “radiopharmaceutical composition” is defined in the present invention as a formulation comprising the radioiodinated compound of the present invention together with a biocompatible carrier in a form suitable for mammalian administration, preferably administration to humans.

The “biocompatible carrier” is a fluid, especially a liquid, in which the radioiodine-containing compound as defined herein is suspended or dissolved, such that the radiopharmaceutical composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The radiopharmaceutical composition may be administered parenterally, i.e. by injection, and is most preferably an aqueous solution. Such a composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid).

### Kit and Cassette

The method of the invention may conveniently be carried out by means of a kit. Therefore, in another aspect, the present invention provides a kit for carrying out the method of the invention, as suitably and preferably defined herein, wherein said kit comprises:
(i) a first vessel comprising either the compound of Formula II as defined herein, or the compound of Formula IIa as defined herein; and,
(ii) a second vessel comprising either the compound of Formula III as defined herein, or the compound of Formula IIIa as defined herein.

When the method of the invention is automated, the first and second vessels of the kit may be housed in a disposable or removable cassette designed for use with an automated synthesis apparatus. Therefore, in another aspect, the present invention further provides a cassette for an automated synthesis apparatus comprising the components as defined above for the kit of the invention.

It is particularly convenient to use a kit or a cassette as described above for the preparation of the radiopharmaceutical composition of the invention. Therefore in a preferred embodiment, either the kit or the cassette of the invention is used in the preparation of the radiopharmaceutical composition of the invention as described herein.

In a further aspect, the present invention provides for the use of the kit or of the cassette of the invention to carry out the method of the invention as described herein.

For the kit and the use thereof, the cassette and the use thereof, Formulas I-III and Formulas Ia-IIIa, and preferred embodiments thereof, are as described above for the method of the invention.

### BRIEF DESCRIPTION OF THE EXAMPLES

Unless otherwise specified, all reagents described in the Examples below were obtained from Sigma-Aldrich or BDH.

50 mM pH7.4 phosphate buffer was prepared using 40.5 ml 0.2M Na₂HPO₄+9.5 ml 0.2M NaH₂PO₄ made up to 200 ml with water.

0.2M pH4 ammonium acetate buffer was prepared using 18 ml 0.2M NH₄OAc+82 ml 0.2M HOAc made up to 1 litre with water.

Example 1 is a comparative example describing a route for indirect iodination based on prior art teachings relating to ¹⁸F labelling via conjugation of a HYNIC-functionalised biomolecule with ¹⁸F-fluorobenzaldehyde. Radiochemical purity (RCP) was 15% with the radioiodination step carried out at 70° C.

Example 2 describes an indirect radioiodination method of the invention comprising reaction of a HYNIC type hydrazine, 2-hydrazinopyridine with N-succinimidyl-3-iodo benzoic acid. RCP was 21% at 37° C. The method of this example has the advantage over the prior art method described in Example 1 that it is carried out at physiological temperature, a temperature which is optimal for the preservation of biomolecular structure and function. In addition, the method of Example 2 demonstrated an improved yield over the prior art method of Example 1.

Example 3 describes the synthesis of 3-trimethylstannyl-benzoic acid 2,5-dioxo-pryolidin-1-yl ester, an intermediate used in the method of Example 2,

Example 4 demonstrates that NHS ester reacts more readily with hydrazine groups than with amino groups.

### Abbreviations Used in the Examples

| | |
|---|---|
| Ac | acetyl |
| DCC | dicyclohexylcarbodiimide |
| DMF | dimethylformamide |
| h | hour(s) |
| HPLC | high performance liquid chromatography |
| LCMS | liquid chromatography mass spectrometry |
| Lys | lysine |
| MeOH | methanol |
| min | minute(s) |
| MS | mass spectrometry |
| m/z | mass-to-charge ratio |
| NHS | N-Hydroxysuccinimide |
| NMR | nuclear magnetic resonance |
| RCP | radiochemical purity |
| TFA | trifluoroacetic acid |
| THF | tetrahydrofuran |
| Thr | threonine |
| TLC | thin layer chromatography |
| t_{R} | retention time |
| UV | ultraviolet |

### EXAMPLES

### Comparative Example 1

### Synthesis of N-[1-(4-Iodo-phenyl)-meth-(E)-ylidene]-N′-pyridin-2-yl-hydrazine

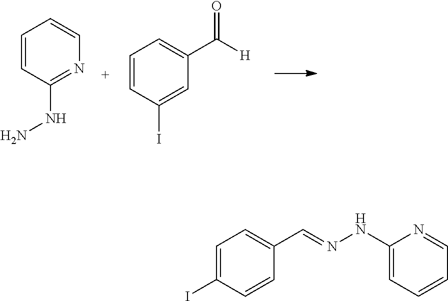

### 1(i) Reaction Carried Out with ¹²⁷I

¹²⁷I conjugation of 4-iodobenzaldehyde and 2-hydrazinopyridine was performed in order to purify the product and analyse by mass spectrometry to confirm its identity.

2-hydrazinopyridine and 4-iodobenzaldehyde were dissolved in ethanol to give 9.2 mM and 4.3 mM solutions.

To a 10 ml silanised sealed glass vial the following were added in the given order:—

10 μl 2-hydrazinopyridine (9.2×10⁻⁸ moles)
170 μl 50 mM pH7.4 phosphate buffer
21 μl 4-iodobenzaldehyde (9.1×10⁻⁸ moles)

The preparation was heated at 37° C. for 20 minutes before analysing by HPLC:

| | |
|---|---|
| Column | Phenomenex Luna C18(2) 5μ 4.6 × 150 mm |
| Flow | 1 ml/min |
| Detection | UV 254 nm and Bioscan radio detector |
| Solvent A | 0.1% TFA in water |
| Solvent B | 0.1% in acetonitrile |
| Gradient | 0 min 0% B |
| | 20 min 70% B |
| | 25 min 90% B |
| | 27 min 90% B |
| | 28 min 0% B |
| | 35 min 0% B |

The peak for N-[1-(4-Iodo-phenyl)-meth-(E)-ylidene]-N'-pyridin-2-yl-hydrazine was HPLC purified. Mass spectrometry showed a peak at 324 which is the correct mass for the expected product.

### 1(ii) Reaction Carried Out with ¹²³I

2-hydrazinopyridine was dissolved in ethanol to give a 9.2 mM solution. 5 μl of peracetic acid was diluted in 5 ml water to give a 5 mM solution.

### Synthon Preparation

37 μl 2.8 mM 4-tri-n-butyltinbenzaldehyde (prepared according to the method of Sessler et al J Am Chem Soc 1995; 117(2): 704-14) in ethanol (1.0×10⁻⁷ moles) was added to a silanised sealed 1.5 ml ‘V’ shaped vial. 200 μl 0.2M pH4 ammonium acetate buffer and 10 μl 1 mM Na¹²⁷I in 0.05M NaOH (1.0×10⁻⁸ moles) were added to a ¹²³I vial. 10 μl 5 mM peracetic acid was added to the ¹²³I vial. The contents of the ¹²³I vial were transferred to the silanised 1.5 ml ‘V’ shaped vial.

### Conjugation

4.4 μl 2-hydrazinopyridine (4.0×10⁻⁸ moles), 100 μl crude 4-iodobenzaldehyde and 100 μl 50 mM pH7.4 phosphate buffer were added to a silanised sealed 10 ml glass vial and the prep heated at 70° C. for 10 minutes.

HPLC, carried out as per Example 1, showed 15% conjugate at t_{R} 14.1 min. There was 38% iodide and 47% synthon remaining.

### Example 2

### Synthesis of 3-iodo-benzoic acid N′-pyridin-2-yl-hydrazide

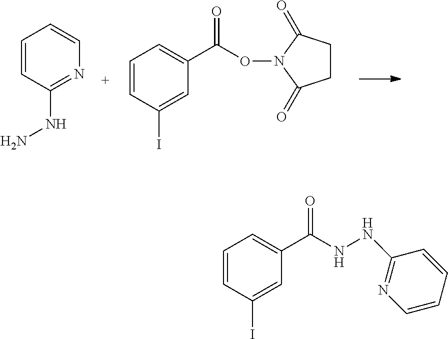

### 2(i) Reaction Carried Out with ¹²⁷I

3-iodo-benzoic acid 2,5-dioxo-pyrrolidin-1-yl ester was prepared by reaction of 3-iodo benzoic acid (1 g, 4 mmol), N-hydroxysuccinimide (464 mg, 4 mmol) and dicyclohexylcarbodiimide (DCC) (4 mls of a 1M solution in dichloromethane, 4 mmol). The reaction mixture was stirred at room temperature for 6 hours in 10 ml DMF The resulting white precipitate was filtered and discarded and the resulting filtrate reduced in vacuo and purified by column chromatography giving a 29% yield.

¹²⁷I conjugation of 3-iodo-benzoic acid 2,5-dioxo-pyrrolidin-1-yl ester and 2-hydrazinopyridine was performed in order to purify the product and analyse by mass spectrometry to confirm its identity.

2-hydrazinopyridine was dissolved in ethanol to give a 9.2 mM solution.

To a 10 ml sealed silanised glass vial the following were added in the given order:—

20 μl 2-hydrazinopyridine (1.8×10⁻⁷ moles)
180 μl 50 mM pH7.4 phosphate buffer
63 μl 2.9 mM 3-iodo-benzoic acid 2,5-dioxo-pyrrolidin-1-yl ester (1.8×10⁻⁷ moles)

The preparation was heated at 37° C. for 30 minutes before analysing by HPLC as described above for Example 1. The peak for 3-iodo-benzoic acid N′-pyridin-2-yl-hydrazide was HPLC purified and analysed by mass spectrometry. Mass spectrometry showed a peak at 340 which is the correct mass for the expected product.

### 2(ii) Reaction Carried Out with ¹²³I

### 2(ii)(a) Synthon Preparation

2-hydrazinopyridine was dissolved in ethanol to give a 17 mM solution. 10 μl of peracetic acid was diluted in 5 ml water then 100 μl of this solution diluted to 1 ml with water to give a 1 mM solution. 3-trimethylstannyl-benzoic acid 2,5-dioxo-pryolidin-1-yl ester (synthesis described in Example 3 below) was dissolved in 1% acetic acid in methanol to give a 0.26 mM solution. A C18 SepPak was conditioned with 5 ml acetonitrile followed by 10 mls water.

38 μl 3-trimethylstannyl-benzoic acid 2,5-dioxo-pyrrolidin-1-yl ester solution (1.0×10⁻⁸ moles) was added to a silanised sealed 1.5 ml ‘V’ shaped vial.

200 μl 0.2M pH4 ammonium acetate buffer and 10 μl 1 mM Na¹²⁷I in 0.05M NaOH (1.0×10⁻⁸ moles) were added to a ¹²³I vial.

The contents of the ¹²³I vial were transferred to the silanised 1.5 ml ‘V’ shaped vial.

The crude synthon was loaded on the pre prepared C18 Sep-Pak. Iodide was eluted with 5 ml water. The synthon was eluted with 2.5 ml acetonitrile into a silanised vial. The acetonitrile was removed under high vacuum.

### 2(ii)(c) Conjugation

6 μl 2-hydrazinopyridine (1.0×10⁻⁷ moles) and 194 μl 50 mM pH7.4 phosphate buffers were added to the dried synthon and the prep heated at 37° C. for 30 minutes.

HPLC showed 21% product at t_{R} 11.3 min. There was 8% synthon left in the crude reaction.

### Example 3

### Synthesis of 3-trimethylstannyl-benzoic acid 2,5-dioxo-pryolidin-1-yl ester

### 3(i) Methyl 3-Iodobenzoate (2)

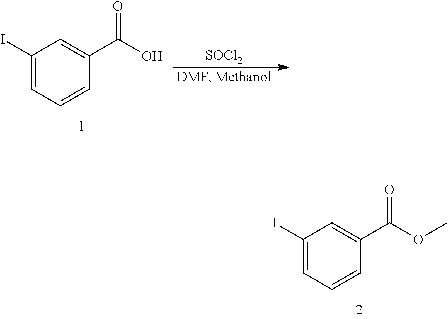

One drop of dimethylformamide was added to a stirring suspension of 3-iodobenzoic acid (1.0 g, 4.03 mmol), in thionyl chloride (20 ml). The mixture was then heated at 80° C. for 18 hours. After cooling to ambient temperature, the solvent was completely evaporated, then methanol (20 ml) was slowly added and the mixture stirred at ambient temperature for 30 minutes. Evaporation of methanol afforded the crude product as oil which solidified on standing at ambient temperature. The crude product was purified by flash chromatography using ethyl acetate/hexane (1:1) and the pure product was obtained as slightly yellow crystals (824 mg, 78.4%).

¹H NMR (500 MHz, CDCl₃, δ 3.95 (s, 3H, CH₃), 7.20 (t, 1H), 7.9 (d, 1H), 8.0 (d, 1H), 8.20 (s, 1H)

### 3(ii) 3-Trimethylstannyl-benzoic acid methyl ester (3)

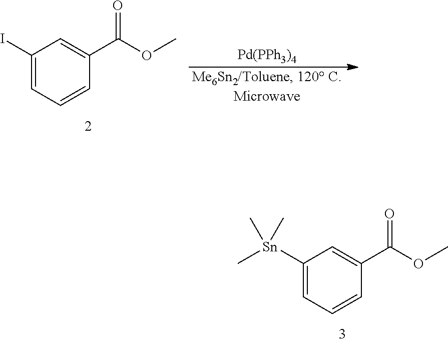

To a mixture of methyl 3-iodobenzoate 200 mg, 0.76 mmol), and hexamethylditin (643 μl, 3.1 mmol) in toluene (8 ml) was added tetrakis (triphenylphosphine) palladium (0) (180 mg, 0.155 mmol (10 mol %). The reaction tube was flushed with argon, capped and heated in a microwave at 120° C. for 15 minutes. After cooling, TLC (ethyl acetate/hexane, 1:1) showed complete conversion to product. Then the black suspension was filtered (filter paper) and the resulting dark coloured solution was evaporated to dryness. The residue was dissolved in ethyl acetate (15 ml) and extracted with water (6×10 ml).

Then the organic phase was dried (MgSO₄) filtered and evaporated under reduced pressure to afford the crude product as a dark oil (463 mg) which was purified by flash chromatography (Ethyl acetate:hexane, 1:1) to afford a slightly yellow oil. Yield: 344 mg (74%).

NB: Microwave irradiation was carried out in a Personal Chemistry Emrys synthesizer.

¹H NMR (500 MHz, CDCl₃, δ 0.32 (s, 9H, Sn(CH₃)₃, 3.95 (s, 3H, CH₃), 7.40 (t, 1H), 7.69 (d, 1H), 8.0 (d 1H), 8.20 (s, 1H)

3-Trimethylstannyl-benzoic acid (4)

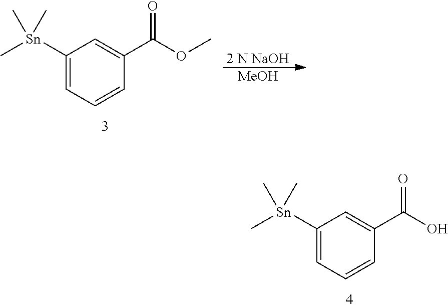

3-Trimethylstannyl-benzoic acid methyl ester (148 mg, 0.50 mmol) was dissolved in methanol (3 ml) and while stirring at ambient temperature, aqueous 2N sodium hydroxide (1 ml, 2 mmol) was slowly added after which the mixture was stirred at this temperature for 3 hours when monitoring by HPLC indicated that the hydrolysis was completed. Then the mixture was evaporated to dryness and the residue re-dissolved in water (4 ml) and acidification by slow addition of 1M hydrochloric acid resulted in precipitation of the product. The mixture extracted with dichloromethane (3×10 ml) and the combined dichloromethane phases was dried (Na₂SO₄), filtered and evaporated to afford the crude product as a white solid material. It was used in the next step without further purification. Yield: 107 mg (76%).

¹H NMR (500 MHz, CDCl₃, δ0.39 (s, 9H, Sn(CH₃)₃, 7.42 (t, 1H), 7.72 (d, 1H), 8.02 (d. 1H), 8.15 (s, 1H)

### 3(iv) 3-Trimethylstannyl-benzoic acid 2,5-dioxo-pyrrolidin-1-yl ester (6)

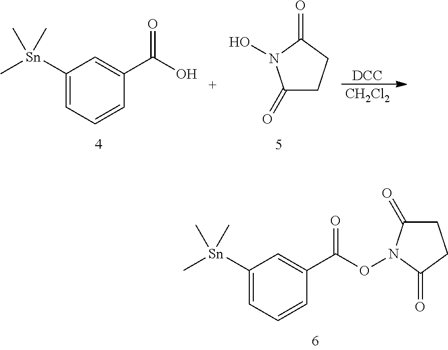

To a solution of 3-trimethylstannyl-benzoic acid (101 mg, 0.35 mmol) in dichloromethane (3 ml) was added N,N′-dicyclohexylcarbodiimide, DCC (72 mg, 0.35 mmol) followed by N-hydroxy-succinimide (40.28 mg, 0.35 mmol). The clear and colourless mixture was left to stir at ambient temperature and a white precipitate started to appear after ca. 15 minutes. The reaction as stopped after 1 hour when TLC (ethyl acetate/hexane, 1:1) showed complete disappearance of the starting material. The mixture was filtered and the residue that was recovered after evaporation of the solvent was purified by flash chromatography using ethyl acetate/hexane, 1:1. The pure product was obtained as colourless oil. Yield: 90 mg (67%).

¹H NMR (500 MHz, CDCl₃, δ 0.25 (s, 9H, Sn(CH₃)₃, 2.88 (broad s, 4H, (CH₂)₂), 7.42 (t, 1H), 7.74 (d, 1H), 8.03 (d. 1H), 8.20 (s, 1H)

### Example 4

### Synthesis of 3-(4-Hydroxy-3-iodo-phenyl)-propionic acid hydrazide

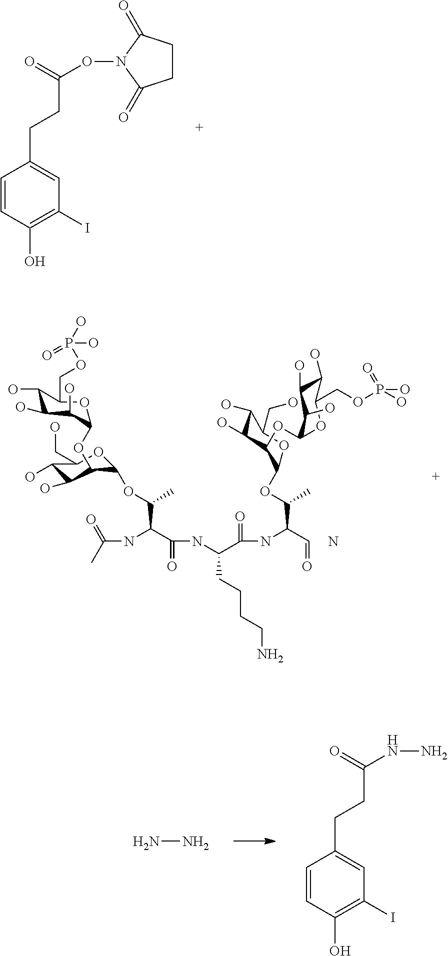

5 mg of Ac-Thr(Mannose-6-Phosphate-Mannose)-Lys-Thr(Mannose-6-Phosphate-Mannose)-NH₂ (“glycopeptide”; synthesized according to methods disclosed by Christensen et al J Chem Soc Perkin Trans 1994; 10: 1299-1310) was reacted with 1.7 mg of iodinated Bolton-Hunter NHS ester (“BH”; Wako pure chemical industries 199-09341) and sym-Collidine (1.1 μL, Fluka 27690) were mixed in 1 ml DMF (Rathburn PTS6020) and the mixture (cloudy solution) was heated at 35° C. for 2 h. All BH was consumed and a new peak corresponding to BH-hydrazine conjugate was observed as sole product. More BH (0.5 mg) and sym-Collidine (1.1 μL) were added and stirring at 35° C. was continued for 2 h. Most BH was consumed and the peak corresponding to BH hydrazine had increased. Acetone (100 μL) was added in order to react with remaining residual hydrazine and the mixture stirred over night at room temperature. More BH (2.0 mg) was then added and stirring continued at 35° C. for 90 min. No consumption of BH was observed indicating that all hydrazine was quenched. More sym-Collidine (2.2 μL) was added and stirring continued at 35° C. for 2 hrs. No product had been formed. Water (500 μL) was added to better dissolve the starting material and stirring continued at 35° C. overnight. Small amount of product was formed. More water (2 mL) and BH (1.8 mg) were added, then THF (2 mL) was added to better dissolve BH, and stirring continued over night. More of the product was formed together with hydrolysed BH. The solvents were partly removed in vacuo and the residue diluted with water/0.1% TFA (8 mL) and subjected to HPLC purification.

Monitoring of the above-described reaction was carried out by LCMS using a Phenomenex Luna 3μ C18 (2) 20×2 mm column, with detection at 214 nm. Solvent A: H₂O/0.1% TFA, solvent B: CH₃CN/0.1% TFA; flow: 0.6 mL/min; gradient: 0-30% B over 5 min, t_{R}: 3.11 min (product); 3.40 min (BH-hydrazine), found m/z: 1471.9 (product); 306.9 (BH-hydrazine), expected MH⁺: 1472.3 (P); 307 (BH-hydrazine).

Purification was carried out by HPLC using a Phenomenex Luna 5μ C18 (2) 250×21.20 mm column, with detection at 214 nm. Solvent A: H₂O/0.1% TFA; solvent B: CH₃CN/0.1% TFA; flow: 10 mL/min; gradient: 0-30% B over 40 min; t_{R}: 36-40 min.

Analysis of purified product was carried out by LCMS using a Phenomenex Luna 3μ C18 (2) 20×2 mm column with detection at 214 nm. Solvent A: H₂O/0.1% TFA; solvent B: CH₃CN/0.1% TFA; flow: 0.6 mL/min; gradient: 0-30% B over 5 min; t_{R}: 3.13 min (P). Found m/z: 1471.9, expected MH⁺: 1472.3. Yield 0.9 mg (15%).

1. A method for the synthesis of a radioiodinated compound of Formula I: 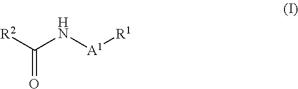
or a salt or solvate thereof, said method comprising reaction of a compound of Formula II: 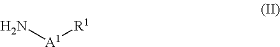
with a compound of Formula III: 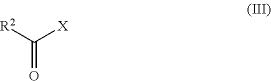
wherein:
A¹ is either NH or O;
one of R¹ and R² is the group -L¹-Ar¹ wherein:
L¹ is a bond or is a bivalent linker comprising 1-3 L* linker units wherein L* is selected from —CO—, —CR′₂—, —CR′═CR′—, —C≡C—, —CR′₂CO₂—, —CO₂CR′₂—, —NR′—, —NR′CO—, —CONR′—, —NR′—, —(C═O)NR′—, —NR′(C═S)NR′—, —SO₂NR′—, —NR′SO₂—, —CR′₂OCR′₂—, —CR′₂SCR′₂—, —CR′₂NR′CR′₂—, a C₅₋₁₂ arylene group, and a C₃₋₁₂ heteroarylene group, wherein R′ is hydrogen or C₁₋₃ alkyl; and,
Ar¹ is a 6-membered C₃₋₆ aryl group, substituted with radioiodine, and with 0-3 other substituents selected from C₁₋₃ alkyl, halo, amino, carboxyl, hydroxyl, or protected versions thereof, and wherein said aryl group has 0-3 heteroatoms selected from N, S and O;
and the other of R¹ and R² is the group -L²-R*, wherein:
L² is a bond or is a bivalent linker comprising 1-6 L* linker units wherein L* is as defined for L¹; and,
R* is a biomolecule;
wherein R¹ and R² optionally comprise suitable protecting groups;
and wherein X represents an active ester group.

2. The method as defined in claim 1 wherein A¹ is NH.

3. The method as defined in claim 1 wherein A¹ is O.

4-5. (canceled)

6. The method as defined in claim 1 wherein R¹ is the group -L²-R* and R² is the group -L¹-Ar¹.

7-8. (canceled)

9. The method as defined in claim 1 wherein:
said compound of Formula I is a compound of Formula Ia: 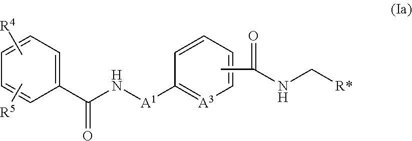
said compound of Formula II is a compound of Formula IIa: 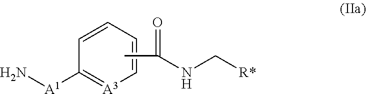
said compound of Formula III is a compound of Formula IIIa: 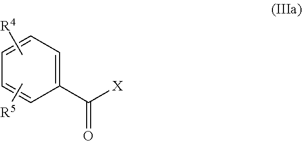
wherein A³ is N or CH, R* is as defined in claim 1, one of R⁴ and R⁵ is radioiodine and the other of R⁴ and R⁵ is hydrogen or hydroxyl, and X is as defined in claim 1.

10. The method as defined in claim 9 wherein A¹ is NH.

11. The method as defined in claim 9 wherein A¹ is O.

12. The method as defined in claim 9, wherein A³ is CH.

13-15. (canceled)

16. The method as defined in claim 1 which is automated.

17. (canceled)

18. A radiopharmaceutical composition comprising the radioiodinated compound of Formula I as defined in the method of claim 1 together with a biocompatible carrier in a form suitable for mammalian administration.

19. A kit for carrying out the method as defined in claim 1 comprising:
(i) a first vessel comprising either the compound of Formula II as defined in the method of claim 1, or the compound of Formula IIa as defined in the method of claim 9; and,
(ii) a second vessel comprising either the compound of Formula III as defined in the method of claim 1, or the compound of Formula IIIa as defined in the method of claim 9.

20. A cassette for carrying out the method as defined in claim 16, said cassette comprising first and second vessels as defined for the kit of claim 19.

21-22. (canceled)
